(19) United States
(12) Patent Application Publication
(10) Pub. No.: US 2006/0206098 A1
(43) Pub. Date: Sep. 14, 2006

(54) Low vibration tube

(76) Inventor: Mike Fard, Charlottesville, VA (US)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 11/368,405

(22) Filed: Mar. 7, 2006

Related U.S. Application Data
(60) Provisional application No. 60/661,014, filed on Mar. 14, 2005.

(51) Int. Cl. A61M 1/00

(52) U.S. Cl. 604/542

(57) ABSTRACT

Energy absorption tubing is provided between the cannula and the suction tubing connected to a vacuum in a Power Assisted Lipoplasty device. The energy absorption tubing isolates the vibration or keeps the motion away from the handpiece, making the handpiece more comfortable to hold for the surgeon.

### PRIORITY INFORMATION

This application claims priority to provisional application Ser. No. 60/661,014 filed Mar. 14, 2005.

### BACKGROUND OF THE INVENTION

1. Field of the Invention

The present invention generally relates to medical devices and, more particularly, to improvements in a Power Assisted Lipoplasty (PAL) instrument.

2. Background Description

Lipoplasty is a surgical procedure in which fat is removed by first inserting a small, hollow tube, called a cannula, through one or more tiny incisions near an area to be suctioned. The cannula is connected by tubing to a vacuum. Guided by the surgeon, the suction device literally vacuums away unwanted fat.

Power Assisted Lipoplasty (PAL) is a technique for liposuction approved by the FDA in December of 1998. With Power Assisted Lipoplasty, the cannula is powered by gas or electricity. The cannula is able to power through tissue and vacuum out the fat and fibrous or scared tissue with less effort than without the power. In traditional lipoplasty, a risk of bruising, swelling and discomfort can accompany the surgery because the surgeon is required to use his/her own strength to move the cannula back and forth.

One of the issues with a Power Assisted Lipoplasty (PAL) instrument is the vibration of the suction tubing due to the reciprocation movement of the cannula. This vibration can be a cause of fatigue for the surgeon during a protracted procedure. Such fatigue is due to the fact that the surgeon grips the handpiece of the device with his or her index finger extending along the front edge of the handpiece. The index finger is very sensitive to this vibration since there are more nerve endings in this finger than in the other fingers.

### SUMMARY OF THE INVENTION

It is therefore an object of the present invention to provide a way to isolate the vibration before it gets to the handpiece therefore making the handpiece more comfortable to hold for the surgeon.

According to the invention, there is provided an energy absorption tubing between the cannula and the suction tubing connected to the vacuum. The energy absorption tubing isolates the vibration or keeps the motion away from the handpiece. In the alternative, the entire length of the suction tubing can be made of an energy absorption tubing.

According to another aspect of the invention, there is provided a disposable package or packages containing either in combination or separately the cannula and the energy absorption tubing.

### BRIEF DESCRIPTION OF THE DRAWINGS

The foregoing and other objects, aspects and advantages will be better understood from the following detailed description of a preferred embodiment of the invention with reference to the drawings, in which:

FIG. 1 is a schematic diagram of a portion of a Power Assisted Lipoplasty (PAL) device showing a conventional connection of the cannula, handpiece and tubing;

FIG. 2 is a schematic diagram showing an improved connection of the cannula to the tubing according to the invention; and

FIG. 3 is a schematic diagram showing an improved connection using silicone tubing according to a preferred embodiment of the invention.

### DETAILED DESCRIPTION OF A PREFERRED EMBODIMENT OF THE INVENTION

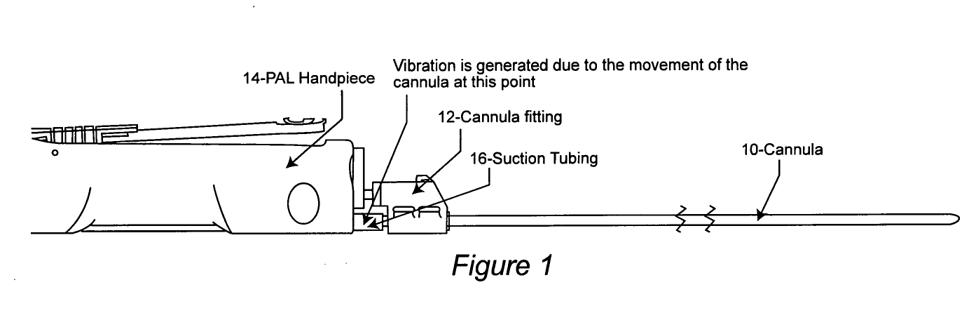

Referring now to the drawings, and more particularly to FIG. 1, there is shown in schematic diagram form the connection of the cannula, handpiece and tubing of a conventional PAL instrument. More particularly, the cannula 10, which is reciprocated by an electrical or pneumatic power source (not shown), is held by a cannula fitting 12 which facilitates attachment to a handpiece 14. The cannula 10 is attached to suction tubing 16 which is connected to a vacuum container. The suction tubing 16, typically made of polyvinyl chloride (PVC), passes through the handpiece 14 but is not attached to the handpiece. An example of this type of PAL instrument is the Power Assisted Lipoplasty devices manufactured and sold by MicroAire® Surgical Instruments of Charlottesville, Va. The MicroAire® Power Assisted Lipoplasty device uses a 2 mm reciprocating movement at 4000 cycles per minute to facilitate the movement of the cannula in tissue.

In use, the surgeon typically holds the handpiece 14 with his or her index finger laying along the front edge of the handpiece. Vibration from the suction tubing 16 is transmitted to the surgeon's index finger. This vibration can be the source of a certain level of fatigue for the surgeon, particularly during protracted surgical procedures.

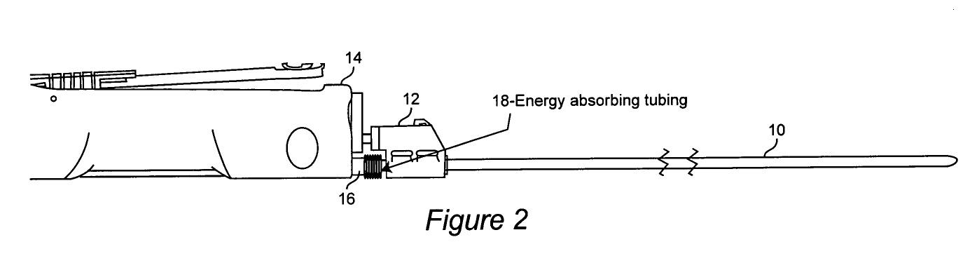

The solution to this problem is generally illustrated in FIG. 2, wherein like reference numerals indicate the same or similar parts as in FIG. 1. Energy absorption tubing 18 is connected between the cannula 10 and the suction tubing 14. The energy absorption tubing 18 is in the form of an accordion or pleated tube and is preferably between 2 mm and 3 mm in length. The energy absorption tubing 18 creates an energy absorption area at the end of the suction tube 16 before it is attached to the cannula 10, thereby isolating the vibration or keeping the motion away from the handpiece.

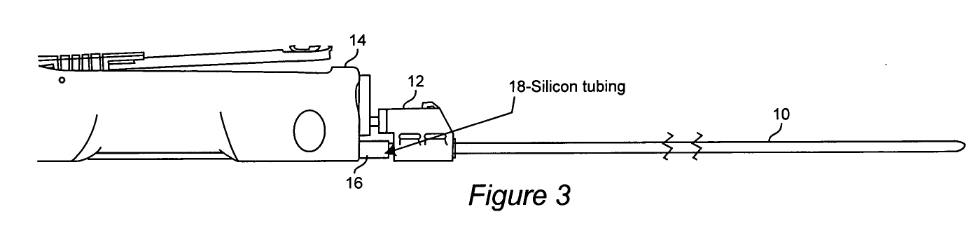

FIG. 3 shows a preferred embodiment of the invention in which the energy absorbing tubing 18 is made of silicone tubing or similar rubbery tubing. The energy absorption tubing 18 can be fitted to the cannula 10 as a unit, and the energy absorption tubing 18 and cannula 10 can be packaged as a complete disposable system. Alternatively, the cannula 10 and the energy absorption tubing 18 can be packaged separately as disposable items. When silicone tubing or similar rubbery tubing is used as the energy absorption tubing, it can be longer than the 2 mm to 3 mm length shown in FIG. 2. For example, the tubing may extend the entire length of the handpiece 14. In some cases, it may be desirable to make the entire length of the suction tube 16 out of silicone tubing or similar rubbery tubing. While silicone tubing is used in the preferred embodiment of the invention, in the alternative, other materials may be used for the energy absorbing tubing 18. One such example is a stretched tube making the tube wall flexible, similarly to the pleated tube shown in FIG. 2. The important thing is that the energy absorption tubing 18 is effective in isolating the vibration from the suction tubing 16 from the handpiece 14.

While the invention has been described in terms of a single preferred embodiment, those skilled in the art will recognize that the invention can be practiced with modification within the spirit and scope of the appended claims.

Having thus described my invention, what I claim as new and desire to secure by Letters Patent is as follows:

1. An improvement in a power assisted lipoplasty device of the type having a reciprocating cannula held by a handpiece, the cannula being connected to a vacuum, the improvement comprising an energy absorption tubing providing a connection between the cannula and the vacuum to thereby isolate the handpiece from vibration from the suction tubing.

2. The improvement in a power assisted lipoplasty device recited in claim 1, wherein the energy absorption tubing is made of a rubbery substance having a flexible sidewall.

3. The improvement in a power assisted lipoplasty device recited in claim 2, wherein in the energy absorption tubing is made of silicone.

4. The improvement in a power assisted lipoplasty device recited in claim 2, wherein the energy absorption tubing connects the cannula to a suction tube.

5. The improvement in a power assisted lipoplasty device recited in claim 4, wherein the energy absorption tubing has a length approximately equal to the handpiece.

6. The improvement in a power assisted lipoplasty device recited in claim 2, wherein the energy absorption tubing serves as a suction tube connecting the cannula to a vacuum.

7. The improvement in a power assisted lipoplasty device recited in claim 1, wherein the energy absorption tubing is made of a stretched tubing so as to have a flexible sidewall.

8. The improvement in a power assisted lipoplasty device recited in claim 7, wherein the energy absorption tubing is between 2 mm and 3 mm in length.

9. The improvement in a power assisted lipoplasty device recited in claim 1, wherein the energy adsorption tubing has a pleated sidewall.

10. The improvement in a power assisted lipoplasty device recited in claim 2, wherein the energy absorption tubing is between 2 mm and 3 mm in length.

11. A disposable package for a power assisted lipoplasty device of the type having a reciprocating cannula held by a handpiece, the cannula being connectable to a vacuum, the disposable package comprising an energy absorption tubing providing a connection between the cannula and the vacuum to thereby isolate the handpiece from vibration from the suction tubing.

12. The disposable package recited in claim 11, wherein the energy absorption tubing is made of a rubbery substance having a flexible sidewall.

13. The disposable package recited in claim 12, wherein the energy absorption tubing is made of silicone.

14. The disposable package recited in claim 11, wherein the energy absorption tubing is made of a stretched tubing so as to have a flexible sidewall.

15. The disposable package recited in claim 11, wherein the energy adsorption tubing has a pleated sidewall.

16. Disposable packages for a power assisted lipoplasty device of the type having a reciprocating cannula held by a handpiece, the cannula being connectable to a vacuum by suction tubing, the disposable packages comprising the cannula in a first package and an energy absorption tubing in a second package, the energy absorption tubing providing a connection between the cannula and the suction tubing to thereby isolate the handpiece from vibration from the suction tubing.

17. The disposable packages recited in claim 16, wherein the energy absorption tubing is made of a rubbery substance having a flexible sidewall.

18. The disposable packages recited in claim 17, wherein the energy absorption tubing is made of silicone.

19. The disposable packages recited in claim 16, wherein the energy absorption tubing is made of a stretched tubing so as to have a flexible sidewall.

20. The disposable packages recited in claim 16, wherein the energy adsorption tubing has a pleated sidewall.
